(19) United States
(12) Patent Application Publication
(10) Pub. No.: US 2019/0030264 A1
(43) Pub. Date: Jan. 31, 2019

(54) Luer Lock Adapter for MDI

(71) Applicant: Disruptive Medical Technologies, LLC, Buffalo Grove, IL (US)

(72) Inventors: Joshua Herskovic, Highland Park, IL (US); Vitaliy Vulykh, Buffalo Grove, IL (US)

(21) Appl. No.: 15/661,502

(22) Filed: Jul. 27, 2017

(51) Int. Cl. A61M 15/00; A61M 39/10; A61M 16/04; A61M 16/10; A61M 16/08; A61M 11/08

(52) U.S. Cl. CPC A61M 15/009; A61M 39/10; A61M 15/0021; A61M 16/0447; A61M 2039/1083; A61M 16/1005; A61M 16/0816; A61M 11/08; A61M 16/0465

(57) ABSTRACT

An improved medical system and user friendly medical device comprises a special Luer type adapter and cap for a metered dose inhaler (MDI) for use with an oxygen delivery circuit and tracheostomy.

### BACKGROUND OF THE INVENTION

This invention relates to a medical dose inhaler (MDI), and more particularly, to a Luer type lock adapter for a MDI and for use with an oxygen delivery circuit and tracheostomy.

Induction of general anesthesia, emergency from anesthesia, emergency intubation of patients in respiratory distress, as well as numerous intraoperative and post-operative events may often result in constriction of bronchi and bronchioles. Such events necessitate administration of bronchodilators such as Albuterol and Ipratropium. While these medications are readily available in operating theaters, critical care units, emergency departments, and ambulances, the administration method of these medications to patients with an artificial airway in place is extremely cumbersome, time-consuming, and with efficacy that often may not be sufficient to achieve desired results.

Currently, such bronchodilators are provided as metered dose inhalers (MDIs), and were designed for patients who are awake, breathing spontaneously, and have enough mental and physical capacity to place the mouthpiece between their lips and press the top of the MDI to receive a dose of the medication upon inhalation.

In the various clinical setting mentioned above, bronchodilators delivered in the form of standard MDIs cannot presently be administered to patients efficiently with an artificial airway such as an endotracheal tube (ETT) or a laryngeal mask airway (LMA), without requiring a MDI delivery system specifically designed for oxygen delivery circuits, since the mouthpiece does not fit the adapter. Non-standard MDIs designed for oxygen delivery circuits cannot be used by patients without advanced airways.

Currently, the available options for administration of MDIs include:

1. Disconnecting oxygen delivery circuit, ventilator, or another oxygen source such as a bag valve mask, placing the MDI over the airway device and delivering a large number of puffs in the hope that an unquantifiable fraction of medication gets delivered to the conducting airways after reconnecting the oxygen source.

2. Dismantling the oxygen delivery circuit and adding an inline circuit adapter that can receive a nebulizer (similar to a standard MDI)

Neither of the two methods mentioned above is reliable, efficient, or particularly effective in delivering medication. Many practitioners have utilized both of these methods in an attempt to deliver the necessary medication and have struggled to obtain satisfactory results.

Currently, there are no effective medical devices which would allow physicians and other practitioners to readily administer bronchodilators to patients with an artificial airway or a tracheostomy via a standard MDI in a reliable, timely, and efficient manner.

It is, therefore, desirable to provide an improved medical system and device, which overcomes most, if not all, of the preceding problems.

### BRIEF SUMMARY OF THE INVENTION

An improved medical system and medical device comprising a Luer type lock adapter, which is also sometimes referred to as a Luer type adapter, Luer type taper adapter, or Luer type slip adapter, is provided for a metered dose inhaler (MDI) for use with an oxygen delivery circuit and tracheostomy. Advantageously, the novel Luer type lock adapter for a MDI provides an effective medical device which would allow practitioners to readily administer bronchodilators via a standard MDI in a reliable, timely, and efficient manner. Desirably, the improved medical system accommodates bronchodilators delivered in the form of MDIs to be administered to patients with an artificial airway such as an endotracheal tube (ETT) or a laryngeal mask airway (LMA), since the mouthpiece of the MDI can readily fit the novel Luer Lock type adapter. The user friendly Luer type adapter for a MDI is a lightweight, portable, single-patient use piece with two sides. One side is arranged and designed to adapt to the Luer type connection on an oxygen delivery system, while the other side is arranged and designed to be attached to the MDI mouthpiece.

The improved medical system, can include: (a) a medication container containing medication, such as a medication canister with a drug vial, and (b) a medical dose inhaler (MDI). The MDI can have a plastic holder comprising a MDI body with a body-passageway. The MDI body can have a MDI inlet for receiving the medication container or canister and can have a mouthpiece-supporting surface. The MDI can also have a mouthpiece connected to and extending from the mouthpiece-supporting surface of the MDI body. The mouthpiece can have an exterior mouthpiece surface and an interior mouthpiece surface, as well as define a mouthpiece-passageway providing a MDI outlet in fluid communication with the MDI inlet and the body-passageway for passage of medication.

The reliable effective Luer type adapter provides an effective medical connection device which features a special MDI-engaging cap. The cap can include a tubular cap-body comprising an inner tubular MDI-engaging end for engaging and receiving the mouthpiece of the MDI. The MDI-engaging end can have an exterior cap-surface and an interior cap-surface. The cap can also have an outer tubular standard artificial airway adapter end providing an outer connector for engaging and connection to an endotracheal tube (ETT), laryngeal mask airway (LMA), or tracheostomy tube. An elongated tubular stem can extend though the cap and can be coaxially surrounded by the tubular cap-body. The stem can have an inner stem-end and define a stem passageway in fluid communication with the mouthpiece-passageway for receiving the medication. Ribs can extend between and connect the stem to the cap-body to position the stem along an axis of the cap. The inner tubular stem can engage a recess surrounding the MDI nozzle to create a more effective seal. The nozzle face can snugly receive and engage the stem. The seal can be improved by adding an elastomeric interface such as an O-ring between the engaging stem and nozzle.

A Luer type connector can be connected to the Luer-lock engaging end of the stem for connection to an oxygen delivery circuit. Preferably, the Luer lock type connector defines a connector-passageway in fluid or pneumatic communication with the elongated stem passageway.

In a preferred embodiment, the MDI engaging cap is comprised of a single unitary piece injection mold medical grade plastic. Alternatively, it may be comprised of other medical grade materials such as stainless steel.

In a variant embodiment, the outer tubular standard artificial airway adapter of the cap can comprise or be attached to an elastomeric material or silicon for providing a gasket and/or seal for sealingly engaging the endotracheal tube, laryngeal mask airway (LMA), or tracheostomy tube.

The inner end and/or outer end of the cap can also have pressure relief openings for enhancing removal of the cap from the MDI, endotracheal tube or tracheostomy tube. The pressure relief openings can comprise perforations or apertures for creating a venturi like effect. Furthermore, the pressure relief openings allow a conscious patient that is spontaneously breathing to draw in surrounding air during application. The pressure relief openings can also comprise slits or circular holes.

In a variant embodiment, the MDI-engaging end can comprise an elastomeric material or silicon for providing a gasket and/or a seal for sealing engaging the MDI mouthpiece. Furthermore, the gasket can allow for the MDI cap to provide a seal against the mouthpiece of MDIs of various shaper and size, thereby providing a universal adapter compatible with multiple MDIs.

A more detailed explanation of the invention is provided in the following detailed descriptions and appended claims taken in conjunction with the accompanying drawings.

### BRIEF DESCRIPTION OF THE DRAWINGS

FIG. 1 is a perspective view of a medical system with a standard medical dose inhaler and a medication canister with a drug vial.

FIG. 2 is a perspective view of the medical system with a Luer type adapter connected to the mouthpiece of the medical dose inhaler in accordance with principles of the present invention.

FIG. 3 is a perspective view of the medical system with the Luer type adapter removed and disconnected from the mouthpiece of the medical dose inhaler in accordance with principles of the present invention.

FIG. 4 is an enlarged perspective view of the Luer type adapter of the medical system with in accordance with principles of the present invention.

FIG. 5 is a fragmentary cross-sectional view of the Luer type adapter of the medical system with in accordance with principles of the present invention.

FIG. 6 is a fragmentary cross-sectional view of the Luer type adapter of the medical system with a tracheostomy tube adapter and tracheostomy tube and showing a venturi effect in accordance with principles of the present invention.

FIG. 7 is a perspective view of the medical system with a Luer type adapter connected to the mouthpiece of the medical dose inhaler with a flexible cannula and an endotracheal tube (ETT) for delivery deep into the lungs of a patient in accordance with principles of the present invention.

FIG. 8 is a diagrammatic cross sectional view of a patient for receiving the flexible cannula and ETT of the medical system of FIG. 7.

FIG. 9 is a perspective view of the medical system with a Luer type adapter connected to the mouthpiece of the medical dose inhaler with an endotracheal tube (ETT), standard ETT/airway adapter and an elbow connected to oxygen delivery circuit tubing of an oxygen delivery circuit in accordance with principles of the present invention.

FIG. 10 is a perspective view of the medical system with a Luer type adapter connected to the mouthpiece of the medical dose inhaler and connected to tracheostomy in accordance with principles of the present invention.

FIG. 11 is a fragmentary cross-sectional view of another Luer type adapter of the medical system with a gasket for engaging the mouthpiece of the medical dose inhaler and having no stem in accordance with principles of the present invention.

### DETAILED DESCRIPTION OF THE INVENTION

The following is a detailed description and explanation of the preferred embodiments of the invention and best modes for practicing the invention.

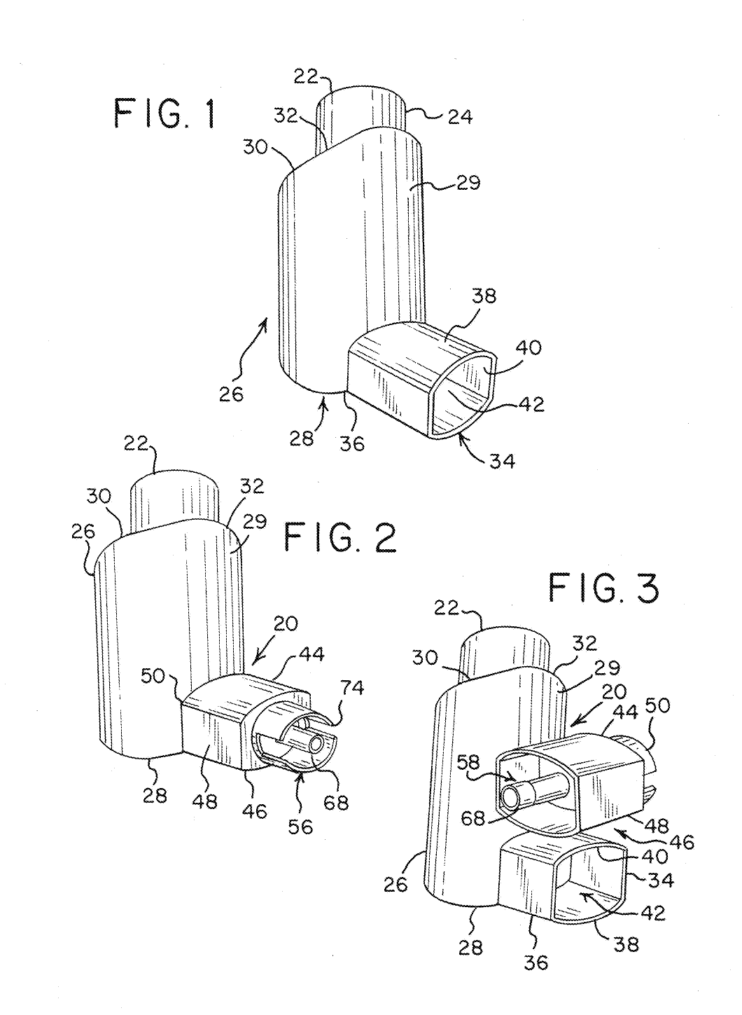

Referring to the drawings, a user friendly medical system 20 (FIGS. 2 and 3) can include an assembly with a medication canister 22 (FIGS. 1-3) that provides a medication container comprising a drug vial 24 (FIG. 1) with medication. The medication canister can fit into and be seated in a plastic holder 26 of a medical dose inhaler (MDI) 28. The MDI can comprise impact-resistant medical grade plastic and have an elongated tubular MDI body 29 providing the plastic holder with an elongated body-passageway 30 and have a medication canister-receiving end 32 providing a MDI inlet for snugly receiving and engaging the medication canister. The MDI can have a tubular mouthpiece 34 integrally connected to and extending at an angle of inclination from the mouthpiece-supporting surface 36 of the MDI body. As shown in FIG. 3, the mouthpiece has an exterior mouthpiece surface 38 and an interior mouthpiece surface 40, as well as can define a mouthpiece-passageway 42 providing a MDI outlet communicating with the MDI inlet 32 and the body-passageway 30 for passage of medication from the drug vial. In the preferred embodiment, body-passageway 30 communicates via mouthpiece-passage 42 to a pin hole size nozzle that the vial engages, so there is no direct communication only indirect communication. In the illustrative embodiment, the MDI body is substantially longer than the mouthpiece.

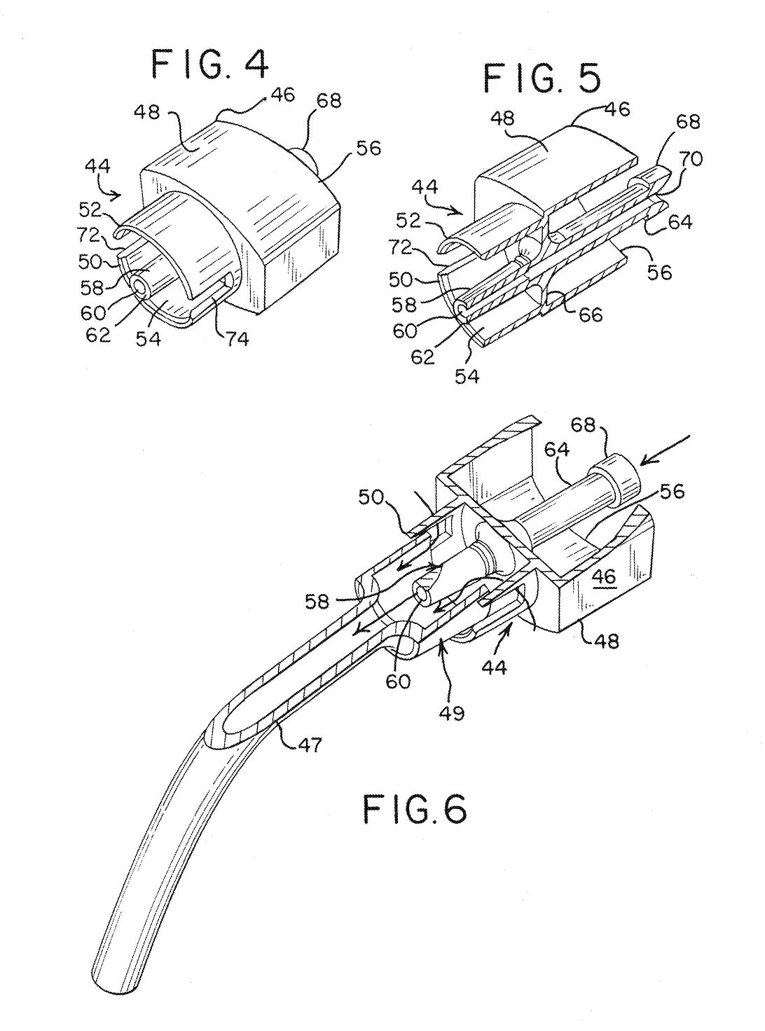

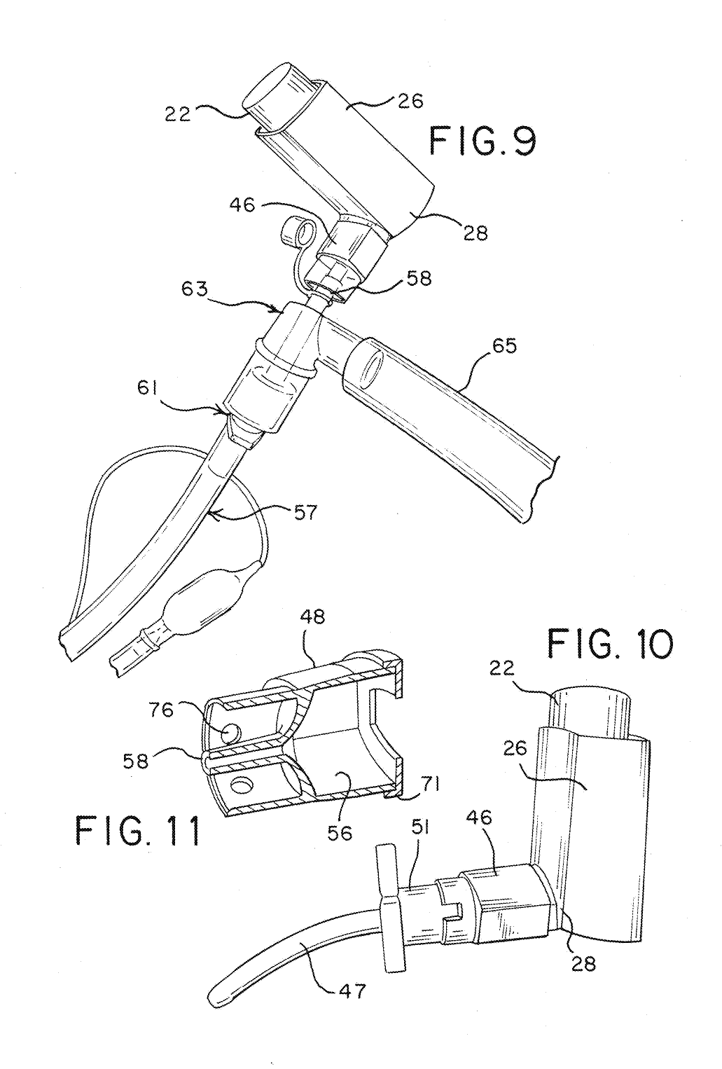

Advantageously, the improved medical system has a user friendly special Luer type lock adapter 44 (FIGS. 2-5) which also is sometimes referred to as Luer type adapter, Luer type taper adapter, or Luer type slip adapter. The Luer type adapter provides a safe easy-to-use light weight medical connection device that features a removable and disposable special MDI-engaging cap 46 comprising a Luer type cap of impact-resistant medical grade fluid-impervious (fluid impermeable) plastic or stainless steel. Desirably, the cap can be used with an intubated patient via an endotracheal tube and/or a patient with a tracheostomy 47 (FIG. 6) inserted via a tracheostomy tube adapter 49 or a tracheostomy connector 51 (FIG. 10). The cap can have a tubular cap-body 48 (FIGS. 4 and 5) comprising an inner tubular MDI-engaging end 56 which engages the mouthpiece. The cap can have an exterior cap-surface 52 and an interior cap-surface 54. The cap can also have an outer tubular standard artificial airway adapter end 50 providing an outer tracheostomy connector for engaging and connection to an endotracheal tube (ETT) 55 (FIG. 9) or to a tracheostomy tube. As shown in FIG. 9, the outer tubular standard artificial airway adapter end and ETT can be connected to a standard ETT/airway adapter 61 through the Luer type lock connector 70 located on the elbow 63 connected to the oxygen delivery circuit tubing 65 of an oxygen delivery circuit to a ventilating machine.

As best shown in FIG. 5, the cap can have an elongated tubular stem 58 that extends axially though the cap. The stem can be annularly and coaxially surrounded by the tubular cap body and can provide an axial elongated stem-passageway 70 which communicates with the mouthpiece-passageway of the mouthpiece of the MDI for receiving medication from the drug vial. The stem can also have an enlarged diameter inner stem-end 68 that engages the MDI mouthpiece and can have an outer Luer lock-engaging end, Luer-engaging end or Luer type engaging end.

One or more transverse radial ribs 66 (FIG. 5) can extend transversely and radially between and integrally connect the stem to the interior cap-surface and cap-body for securely and fixedly positioning the stem along an axis of the cap.

A tubular Luer-type lock connector 58 (FIG. 5), also referred to as a, Luer-type connector, Luer-type taper connector or Luer-type slip connector, terminating in the Luer-type lock engaging end 62 of the elongated tubular stem can connect to the Luer-type connector of 70 for connection to anan oxygen delivery circuit (FIG. 9). The Luer type connector can provide and define a connector-passageway 60 that can be coaxially positioned, and axially aligned and communicate with the stem passageway. The stem functions and acts as a conduit from the MDI outlet to the Luer-type lock connector. The Luer-type lock connector desirably comprises medical grade plastic and allows connection to the oxygen delivery circuit without disconnecting the oxygen delivery circuit. In the illustrative embodiment, the Luer-type lock connector has a maximum axial length that is substantially shorter than an axial stem-length of the stem.

In a variant embodiment the outer tubular standard artificial airway end 56 (FIG. 11) of the cap can comprise an elastomeric material or silicon for providing a gasket 71 and seal for sealingly engaging the mouthpiece of the MDI. Furthermore in the variant embodiment, in lieu of a stem, the fluid is directly channeled from the inner portion of the MDI engaging end 56 thru the Luer-type end 58.

Furthermore, the inner MDI-engaging end of the cap and/or the outer tubular standard artificial airway end of the cap can have pressure relief openings 72 (FIG. 4), such as longitudinal elongated slits 74, for enhancing and facilitating removal of the cap from the MDI mouthpiece, endotracheal tube and/or tracheostomy tube. The pressure relief openings can also comprise perforations, apertures or circular holes 76 (FIG. 11) for creating a venturi like effect.

The novel Luer-type cap can be used for any type of patient, but has the additional advantage over a normal, standard or conventional MDI in that the novel Luer type cap can be used for an intubated patient via endotracheal tube (ETT), laryngeal mask airway (LMA) and a patient with a tracheostomy inserted.

Desirably, the inventive Luer lock type cap permits and facilitates the MDI to be reused between patients (or in the USA with strict OSHA laws reusable with the same patient) since the portion (cap) that interacts with the patient can be discarded and replaced. The novel Luer type cap can be removable and disposable for OSHA/multi-use benefits.

The Luer type connector allows connection to an oxygen delivery circuit without the necessity and need to disconnect the oxygen delivery circuit as is a problem and issue with conventional circuit adapters.

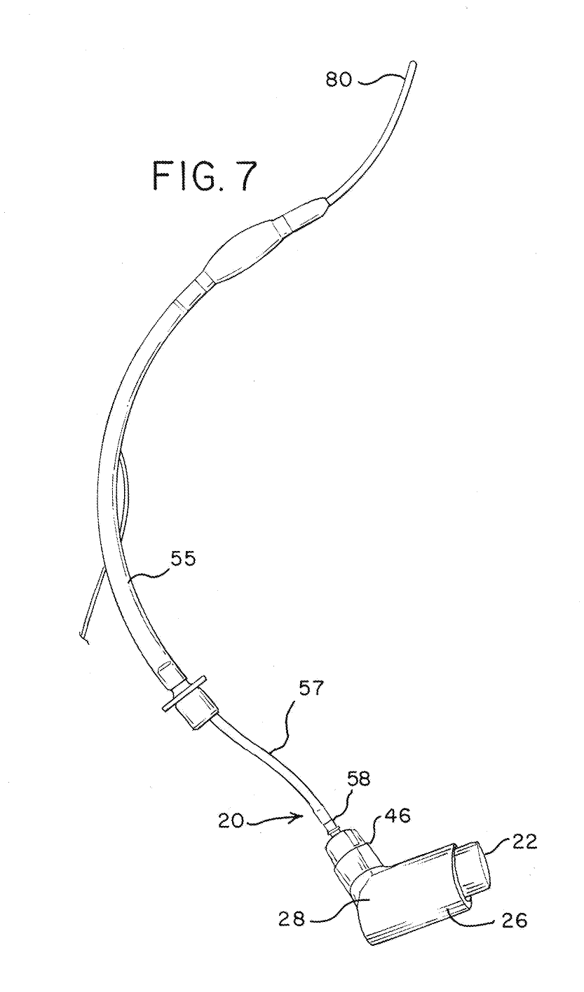

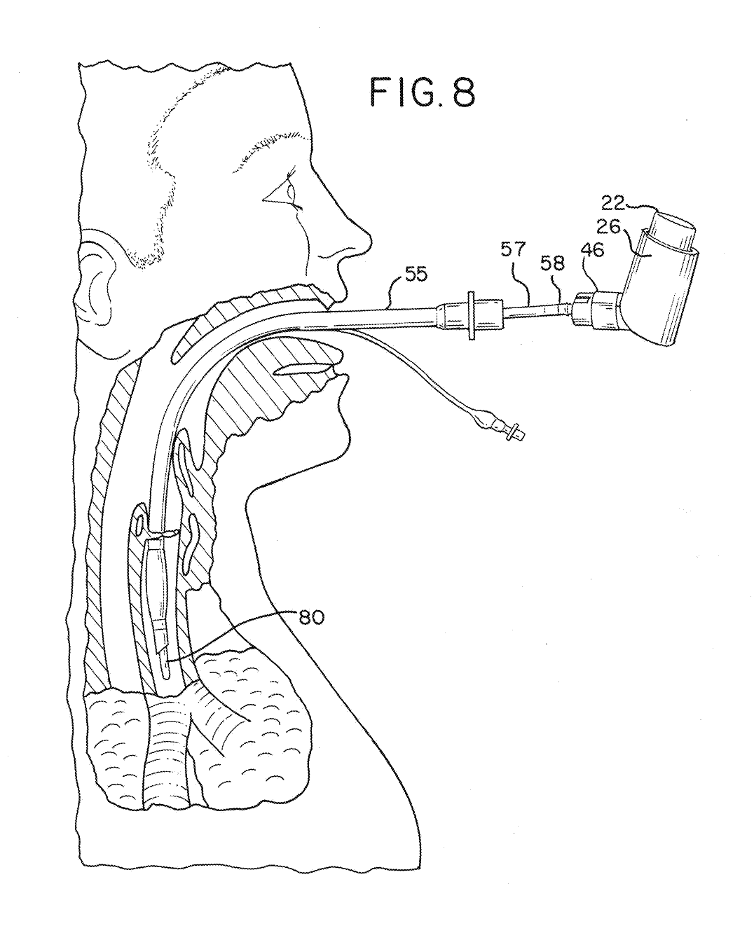

The Luer type connector 58 also allows for connection to other devices, such as a flexible cannula by engaging the flexible cannula Luer type connector portion of the flexible cannula (FIG. 7) to deliver medication to distal portions of the lung via distal tip 80 and bypass the ETT.

The Luer type connector allows the anesthesiologist to connect to the Luer type lock sampling port 70 of the endotracheal tube (ETT) circuit (FIG. 9), This allows for administering drug without interrupting continuous gas flow or ventilation thus more drug should reach the patient.

The stem interacts with outlet on the MDI to minimize pressure loss that occurs. The stem terminates in the Luer type connector.

The user can also connect directly to a tracheotomy via the outer connector on the medical device. The outer connector can also have perforations or a slit opening. The perforations reduce the force needed to remove the device because of the frictional fit. The perforations also allow a conscious patient to draw air without creating a vacuum. Significantly, the perforations allow for a venturi like effect to occur. As the medication exits the centralized Luer type connector, surrounding air will be drawn in to increase drug dispersion (FIG. 6). Also, the Luer type connector provides the additional benefit of directing the gas centrally thru the tracheostomy tube. The outer connector can also be used by a normal patient with the outer connector representing the mouth piece of a MDI.

The slots around the tracheostomy connector can be of any shape such as circular holes or non-existent.

The portion that connects to the MDI can be of various shapes such as a tapered circular opening and be made of a silicone like material to create a gasket like seal. In that way the device can function as a universal adapter for different MDIs. For a universal version fit there can be no stem.

Among the many other advantages of the improved medical system and novel Luer type adapter and cap are:

1. Superior performance.

2. User friendly.

3. Reliable

4. Easy to install and use.

5. Portable.

6. Light weight.

7. Durable.

8. Economical.

9. Safe.

10. Efficient.

11. Effective.

Although embodiments of the invention have been shown and described, it is to be understood that various modifications, substitutions, and rearrangements of parts, components, and/or equipment, as well as other uses, shapes, construction, and design of the improved medical system and novel Luer type adapter and cap can be made by those skilled in the art without departing from the novel spirit and scope of this invention.

What is claimed is:

1. A medical system, comprising:
a medication container containing medication;
a medical dose inhaler (MDI) comprising
a MDI body defining a body-passageway and having a MDI inlet for receiving said medication container and having a mouthpiece-supporting surface; and
a mouthpiece connected to and extending from said mouthpiece-supporting surface of said MDI body and defining a mouthpiece-passageway providing a MDI outlet communicating with said MDI inlet and said body-passageway for passage of medication, and said mouthpiece having an exterior mouthpiece surface and an interior mouthpiece surface; and
a Luer type adapter providing a medical connection device comprising a MDI-engaging cap having
a tubular cap-body comprising a MDI-engaging end for engaging and receiving said mouthpiece;
an outer tubular end providing a connector for engaging and connection to a standard artificial airway adapter, endotracheal tube (ETT), laryngeal mask airway (LMA) or tracheostomy tube;
an elongated tubular stem extending though said cap and coaxially surrounded by said tubular cap-body, said elongated tubular stem defining a stem passageway communicating with said mouthpiece-passageway for receiving the medication, said elongated tubular stem having an inner stem-end and an outer Luer type engaging end;
ribs extending between and connecting said elongated tubular stem to said cap-body for positioning said elongated tubular stem along an axis of said cap; and
a Luer-type connector integrally connected to said Luer type engaging end of said elongated tubular stem for connection to an oxygen delivery circuit and said Luer type connector defining a connector-passageway communicating with said elongated stem passageway.

2. A medical system in accordance with claim 1 wherein at least one of said ends is selected from the croup consisting of said inner MDI-engaging end and said outer tubular end, and comprises silicon for providing a gasket for sealingly engaging the mouthpiece, endotracheal tube (ETT), laryngeal mask airway (LMA), or tracheostomy tube.

3. A medical system in accordance with claim 1 wherein at least one of said ends is selected from the croup consisting of said inner MDI-engaging end and said outer tubular end, and comprises pressure relief openings for enhancing removal of said cap from the MDI, endotracheal tube (ETT), laryngeal mask airway (LMA) or tracheostomy tube.

4. A medical system in accordance with claim 3 wherein said pressure relief openings comprise perforations or apertures for creating a venturi like effect.

5. A medical system in accordance with claim 3 wherein said pressure relief openings comprise elongated slits or circular holes.

6. A medical system in accordance with claim 1 wherein said MDI has a nozzle comprising a pinhole nozzle positioned and surrounded by a recess, and said stem snugly fits in the recess surrounding said nozzle.

7. A medical system, comprising:
a Luer type adapter providing a medical connection device comprising a cap having
a tubular cap-body comprising an inner tubular mouthpiece-engaging end for receiving and engaging a mouthpiece of a medical dose inhaler (MDI), said cap-body having an exterior cap-surface and an interior cap-surface;
an outer tubular end providing a connector for engaging and connection to a standard artificial airway adapter, endotracheal tube (ETT), laryngeal mask airway (LMA) or tracheostomy tube; and
a tubular Luer type connector for connection to a Luer-type connector of an oxygen delivery circuit or a flexible cannula and defining a connector-passageway coaxially positioned and axially communicating with said axial elongated stem passageway.

8. A medical system in accordance with claim 7 wherein at least one of said ends is selected from the group consisting of said inner MDI-engaging end and said outer tubular end, and comprises elastomeric material for providing a gasket and seal for sealingly engaging the mouthpiece, standard artificial airway adapter, endotracheal tube (ETT), laryngeal mask airway (LMA) or tracheostomy tube.

9. A medical system in accordance with claim 7 comprising:
an elongated tubular stem extending axially though said cap and annularly and coaxially surrounded by said tubular cap-body, said elongated tubular stem defining an axial elongated stem passageway communicating with the MDI for receiving medication from a drug vial, said elongated tubular stem having an inner stem-end and an outer end;
at least one rib extending between and connecting said elongated tubular stem to said cap-body for positioning said elongated tubular stem along an axis of said cap; and
said outer tubular endotracheal tube-engaging end comprises a tapered circular opening;
said cap comprises impact-resistant medical grade plastic;
said cap comprises a removable and disposable cap; and
said tubular Luer type connector comprises medical grade plastic and allows connection to the oxygen delivery circuit without disconnecting the oxygen delivery circuit.

10. A medical system in accordance with claim 7 wherein:
at least one of said ends is selected from the group consisting of said inner mouthpiece-engaging end and said outer tubular end, and comprises pressure relief openings for enhancing removal of said cap from the MDI, endotracheal tube (ETT), laryngeal mask airway (LMA) or tracheostomy tube; and
said pressure relief openings are selected from the group consisting of perforations, apertures, slits, and holes.

11. A medical system, comprising:
a medication canister providing a medication container comprising a drug vial with medication;
a medical dose inhaler (MDI) comprising impact-resistant medical grade plastic having
an elongated tubular MDI body defining an elongated body-passageway and having a medication canister-receiving end providing a MDI inlet for snugly receiving and engaging said medication canister and having a mouthpiece-supporting surface; and
a tubular mouthpiece integrally connected to and extending at an angle of inclination from said mouthpiece-supporting surface of said elongated tubular MDI body and defining a mouthpiece-passageway providing a MDI outlet communicating with said MDI inlet and said body-passageway for passage of medication from the drug vial, and said tubular mouthpiece having an exterior mouthpiece surface and an interior mouthpiece surface; and
a Luer type adapter providing a medical connection device comprising a MDI-engaging cap of impact-resistant medical grade plastic or stainless steel having
a tubular cap-body comprising an inner tubular MDI-engaging end for snugly engaging said exterior mouthpiece surface, said tubular cap-body having an exterior cap surface and an interior cap surface;
an outer tubular end providing an outer connector for engaging and connection to a standard artificial airway adapter, endotracheal tube (ETT), laryngeal mask airway (LMA) or tracheostomy tube;
an elongated tubular stem extending axially though said cap and annularly and coaxially surrounded by said tubular cap-body, said elongated tubular stem defining an axial elongated stem passageway communicating with said mouthpiece-passageway for receiving medication from the drug vial, said elongated tubular stem having an inner stem-end and an outer Luer type engaging end;
radial ribs extending radially between and integrally connecting said cap-body for positioning said elongated tubular stem along an axis of said cap; and
a tubular Luer type connector connected to said Luer type engaging end of said elongated tubular stem for connection to an oxygen delivery circuit, and said Luer-type connector defining a connector-passageway coaxially positioned and axially communicating with said axial elongated stem passageway.

12. A medical system in accordance with claim 11 wherein at least one of said ends is selected from the group consisting of said inner mouthpiece-engaging end and said outer tubular end, and is attached to elastomeric material comprising silicon for providing a gasket and seal for sealingly engaging the mouthpiece, endotracheal tube (ETT), laryngeal mask airway (LMA), or tracheostomy tube.

13. A medical system in accordance with claim 11 wherein said outer tubular end comprises a tapered circular opening.

14. A medical system in accordance with claim 11 wherein said inner MDI-engaging end and/or said outer tubular end comprises pressure relief openings for enhancing and facilitating removal of said cap from the MDI, standard artificial airway adapter, endotracheal tube (ETT), laryngeal mask airway (LMA) or tracheostomy tube.

15. A medical system in accordance with claim 14 wherein said pressure relief openings comprise perforations or apertures for creating a venturi like effect.

16. A medical system in accordance with claim 14 wherein said pressure relief openings comprise longitudinal elongated slits.

17. A medical system in accordance with claim 14 wherein said pressure relief openings comprise circular holes.

18. A medical system in accordance with claim 11 wherein said impact-resistant medical grade plastic of said MDI-engaging cap comprises a fluid-impervious plastic for use with an intubated patient via the endotracheal tube and a patient with a tracheostomy inserted.

19. A medical system in accordance with claim 11 wherein:
said MDI-engaging cap comprises a removable and disposable cap; and
said tubular Luer type connector comprises medical grade plastic and allows connection to the oxygen delivery circuit without disconnecting the oxygen delivery circuit.

20. A medical system in accordance with claim 11 wherein:
said elongated tubular MDI body is substantially longer than said tubular mouthpiece; and
said tubular Luer type connector has a maximum axial length substantially shorter than an axial stem-length of said elongated tubular stem.
